(19) United States
(12) Patent Application Publication
(10) Pub. No.: US 2019/0160013 A1
(43) Pub. Date: May 30, 2019

(54) SLOW RELEASE COMPOSITION OF ACTIVE INGREDIENTS

(71) Applicant: Vivek Anand PARACHUR, Chennai (IN)

(72) Inventor: Vivek Anand PARACHUR, Chennai (IN)

(21) Appl. No.: 16/321,152

(22) PCT Filed: Jul. 26, 2017
(86) PCT No.: PCT/IN2017/050306

§ 371 Date: Jan. 28, 2019

(30) Foreign Application Priority Data
Jul. 26, 2016 (IN) 201641025485

(51) Int. Cl. A61K 9/28; A61K 9/20; A61K 31/17

(52) U.S. Cl. CPC A61K 9/2893; A61K 9/288; A61K 31/17; A61K 9/2009; A61K 9/286

(57) ABSTRACT

The present invention discloses a formulation for slow release of at least one active ingredient, wherein the formulation comprises of at least one active ingredient, at least one matrix forming or coat forming natural compound/component and at least one hardening agent. The formulation of the invention may further comprise an acid.

### FIELD OF INVENTION

The present invention relates to a composition for slow or sustained or controlled release of at least one active ingredient. More particularly, the invention relates to a composition containing slow release encapsulation matrix which comprises of a matrix forming natural compound and a hardening agent.

### BACKGROUND OF INVENTION

Oral drug delivery is a time tested route for delivery of drugs and has been proved to be very effective and economical for delivery of majority of drugs in use today. In case of diseases which require more frequent administration of drug, the preferred oral dosage form is a slow or sustained release tablet. The sustained release formulations are designed in such a manner that the tablet releases the drug(s) over a time in the GI tract, such that the drug is released slowly and steadily into the bloodstream.

Conventionally, the sustained-release tablets are formulated such that the active ingredient(s) is (are) embedded in a matrix of insoluble substances such that the dissolving drug must first find its way out through the pores of the matrix before it could get absorbed into the body. The matrix may be a hydrophobic matrix or a hydrophilic matrix or a lipid matrix or a metal matrix or biodegradable matrix. Most of the matrixes used in these conventional sustained release formulations are synthetic in nature.

Use of natural compounds for pharmaceutical applications is attractive because they are economical, readily available and non-toxic. They are capable of chemical modifications, potentially biodegradable and with few exceptions, also biocompatible. Additionally, the natural polymers show no adverse effect on the environment or human beings or animals.

Therefore, the present inventors explore a possibility of using natural compounds or natural compound containing components, which are procured from number of plants or animals, as a probable alternative for use of synthetic polymers for forming a sustained release matrix for slow or sustained or controlled release of pharmaceutical, nutraceutical and nutritional active ingredients.

### SUMMARY OF THE INVENTION

In an aspect the invention provides a slow or sustained or controlled release matrix composition comprising at least one active ingredient, at least one matrix forming natural compound or natural compound containing component, at least one hardening agent and, optionally, at least one acid.

The matrix forming natural compound or natural compound containing component may be selected from oleoresins, oleo gum resins, gum resins, rosins, lipids, fats, free fatty acids, fatty acid distillates, fatty acid residues, sludge oil, fatty acid derivatives, spent liquors, mother liquors and/or wax. The hardening agent is selected from metal hydroxides, metal oxides, metal carbonates, metal chlorides, metal chelates and/or metal phosphates. The acid may optionally be selected from an organic acid or an inorganic acid.

The active ingredient(s) may be selected from pharmaceuticals, biologics, nutraceuticals, nutritional supplements, veterinary drugs, veterinary supplements, veterinary additives, food supplements, food additives or agricultural ingredients.

In another aspect, the invention provides a process for preparation of the sustained release matrix composition.

### BRIEF DESCRIPTION OF DRAWINGS

FIG. 1 illustrates a graph depicting amount of urea released over a span of 8 hours, from the formulation of Example 17.

FIG. 2 illustrates a graph depicting amount of lutein released over a span of 8 hours from the formulation as disclosed in Example 2.

FIG. 3 illustrates a graph depicting amount of caffeine released over a span of 8 hours from the formulation as disclosed in Example 4.

FIG. 4 illustrates a graph depicting amount of Vitamin B6 (pyridoxine) released over a span of 8 hours from the formulation as disclosed in Example 5.

FIG. 5 illustrates a graph depicting amount of Vitamin C (Ascorbic acid) released over a span of 8 hours from the formulation as disclosed in Example 6.

FIG. 6 illustrates a graph depicting amount of Zeaxanthin released over a span of 8 hours from the formulation as disclosed in Example 7.

FIG. 7 illustrates a graph depicting amount of Sylimarin release over a span of 8 hours from the formulation as disclosed in Example 8.

FIG. 8 illustrates a graph depicting amount of Chlorogenic acid release over a span of 8 hours from the formulation as disclosed in Example 9.

FIG. 9 illustrates a graph depicting amount of Bacosides release over a span of 8 hours from the formulation as disclosed in Example 10.

FIG. 10 illustrates a graph depicting amount of Policosanals release over a span of 8 hours from the formulation as disclosed in Example 11.

FIG. 11 illustrates a graph depicting amount of Astaxanthin release over a span of 8 hours from the formulation as disclosed in Example 12.

### DETAILED DESCRIPTION OF INVENTION

The present invention describes a slow release composition that comprises an active ingredient, a matrix forming or coat forming natural compound or natural compound containing component, a hardening agent and, optionally, an acid.

For purposed of this invention, the term “hardening agent” and plurals thereof covers metal oxides, metal hydroxides, metal carbonates, metal chlorides and metal phosphates.

For the purpose of this intention term “natural compound” also encompasses natural compound containing component derived from natural source.

In an embodiment, the matrix forming natural compound is selected from oleoresins, oleoresin spent, oleo gum resins, gum resins, resins, rosins, lipids, fats, fatty acids, fatty acid distillates, spent liquors, mother liquors and/or a wax or by-products of food and agricultural industries.

In accordance with the above embodiment, the oleoresin, oleo gum resin, gum resin, fatty acid distillates, wax or rosin is a crude or fractionated extract/by-product of a plant selected from, but not limited to, Curcuma longa, Boswellia serrata, Bacopa monnieri, Marigold, Ginger, Glycyrrhiza glabra, Cinnamon species, Terminalia chebula, Scutellaria baicalensis, Pinus pinaster (Maritime pine bark), Euterpe oleracea, Acacia catechu, Silybum marianum, Viscum album, Punica granatum, Camellia sinensis (Green Tea), Green coffee bean, Commiphora (Mukul), Cassia fistula, Carica papaya, Centella asiatica, Cinnamomum zeylanicum, Cissus quadrangularis, Chlorophytum tuberosum, Curcuma zedoaria, Curcuma xanthorrhiza, Emblica officinalis, Eugenia jambolana, Eurycoma longifolia Root, Garcinia cambogia, Garcinia mangostana, Gymnema sylvestre, Indigofera tinctoria, Momordica charantia Fruit (Chamomile), Morinda citrifolia, Moringa oleifera, Mucuna pruriens, Piper nigrum Fruit, Phyllanthus niruri, Salacia oblonga, Salacia reticulata, Sphaeranthus indicus, Sida cordifolia, Tagetes erecta Flower, Tamarindus indica, Terminalia arjuna, Terminalia chebula, Tribulus terrestris, Trigonella foenum-graecum, Triphala, Ashwagandha, Resverarol, Hupericin, Acai, Bilberry, Raspberry, Cranberry, Grape Seed, Monagosteen, Noni, Olive, Pomegranate, Beet root, Copal resin, Commiphora myrrha resin, Pine resin, palm fatty acid distillate, sunflower fatty acid distillate, coconut oil sludge, coconut oil fatty acid distillate, rice bran wax, petroleum wax, paraffin wax, etc.

Preferably, the matrix forming natural compound is selected from Turmeric Oleoresin, Boswellia Oleo Gum Resin, turmeric oleoresin spent, fatty acid distillates, wax, selected from vegetable wax, rice bran wax, sunflower oil wax, carnauba wax, shellac wax and the like either alone or in combination.

Accordingly, non-curcuminoid component in the Turmeric Oleoresin is in the range of 5% to 100%. Further, the Turmeric Oleoresin may be selected from, but not limited to, Turmeric Oleoresin, Turmeric oleoresin Spent or Mother Liquor.

The matrix forming natural compound can be either in purified, semi-purified or crude extracts or by-product.

In accordance with the above embodiment, the lipid component is selected from, but not limited to, by-products of oil refineries and phytochemical purifying industry such as Oleoresin Spent, Fatty Acids, Free Fatty Acid Distillate, Palm Fatty Acid Distillate, fatty acid residues, sludge oil, fatty acid derivatives, spent liquors, mother liquors, palm stearin, Omega 3 fatty acids, hydrogenated vegetable oil, vegetable Waxes and other lipid components.

In subsequent embodiment, the hardening agent is selected from, but not limited to, metal hydroxides, metal oxides, metal chlorides and metal carbonates such as Calcium Hydroxide (Ca(OH)₂)/Calcium Oxide, Magnesium hydroxide (Mg(OH)₂)/Magnesium Oxide (MgO), Magnesium Chloride (MgCl₂), Zinc Hydroxide (Zn(OH)₂)/Zinc Oxide (ZnO) and Iron hydroxide (Fe(OH)₂)/Iron Oxide (FeO), Calcium Chloride (CaCl₂)/Calcium carbonate (CaCO₃), Magnesium chloride (MgCl₂)/Magnesium carbonate (MgCO₃), dicalcium phosphate, sodium hydroxide, potassium hydroxide. The most preferred hardening agents being Calcium Hydroxide (Ca(OH)₂) and Magnesium Hydroxide (Mg(OH)₂) used either alone or in combination.

The matrix forming natural compound forms a hard matrix upon reaction with hardening agents and, optionally, acids. While hardening, the matrix embeds the active ingredients, which results in slow or sustained release of the active. Alternatively, Matrix can be coated first followed by hardening agent to coat the active ingredients.

In an optional embodiment, the slow or sustained or controlled release composition contains an acid selected from, but not limited to, propionic acid, citric acid, fumaric acid, tartaric acid, adipic acid, acetic acid, succinic acid, maleic acid, hydrochloric acid and phosphoric acid.

In further embodiment, the sustained release matrix composition of the invention may, optionally, contain pH modifiers including but not limited to succinic acid, maleic acid, humic acid, fumaric acid, tartaric acid, adipic acid, acetic acid, buffers such as phosphate buffer, acetate buffer. The acid may be added to the matrix to increase the stability if there is any stability issue due to pH of the matrix.

In an embodiment, in the sustained release composition, the concentration of sustained release matrix is in the range of 5 to 99% and the concentration of active ingredient(s) is in the range of 1 to 90%.

In an embodiment, the active ingredients are in solid (powder, granules, crystals), semisolid or in liquid form and are selected from, but not limited to, pharmaceuticals, phytochemicals, phytochemical containing components, nutraceuticals, natural extracts, vitamin(s), metal(s), animal extract(s), food ingredient(s), beverage ingredient(s), agrifood ingredient(s) and agricultural ingredient(s).

The pharmaceutical ingredients are selected from, but not limited to all forms of active pharmaceutical ingredients such as paracetamol, ibuprofen, colesevalam, antibiotics such as doxycycline and drugs requiring sustained release profile to achieve therapeutic benefits or to reduce the associated toxicity.

The phytochemical containing component is selected from, but not limited to, solvent extract containing polyphenols, phenolic acids, flavonoids, Terpene, Sesquiterpenes, terpinoids, plant sterols, tannins, alkaloids, carotenes, pterostilbenes, ketones, quinones, amino acids, peptides, either alone or in combination.

The phytochemical is selected from, but not limited to, polyphenols, phenolic acids, flavonoid, Terpene, terpinoids, plant sterols, tannins, alkaloids, carotenes, pterostilbenes, quinones, amino acids, peptides either alone or in combination.

The phytochemical is selected from all hydrophobic and hydrophilic natural compounds, but not limited to, Lutein, Caffeine, Resveratrol, Berberin, 95% Curcuminoids, Gingerols, Bacosides, Boswellic Acids, Chlorogenic Acids, Xanthophils, Astaxanthin, Zeaxanthin, Fucoxanthin, Quercitin, Policosonals, Silymarin, Baicalin, Pycnogenol, Coenzyme Q10, Tocopherols either alone or in combination.

The vitamins are selected from but not limited to water soluble and fat soluble vitamins such as Vitamin A, Vitamin B (B1, B2, B3, B5, B6, B7, B9, B12), Vitamin C, Vitamin E, Vitamin D, Vitamin K1, Vitamin K2, Vitamin K7, Folic acid either alone or in combination.

The metals are selected from but not limited to Calcium, Magnesium, Zinc, Iron, Selenium, Chromium, Manganese, Iodine, Cobalt, Copper, Phosphorous and their salts either alone or in combination.

The said active ingredients are selected from MSM, amino acids, Glucosamine, chondroitin sulphate, alpha lipoic acid, omega 3 fatty acids, omega 6 fatty acids, MCTs, choline, Niacin, L-arginine, acetyl, L-carnitine, Glutathione.

The said veterinary ingredient are selected from but not limited to Sodium Butyrate, Iodine, urea, lysine, methionine, all trace metals and other metals, vitamins, drugs either alone or in combinations.

The said agricultural ingredients are selected from but not limited to urea, pesticides, insecticides, metals and fertilizers.

In an embodiment, the active ingredients may either be solubilized in the sustained release matrix or suspended uniformly in the matrix or single/double coated with the matrix preferably, the active ingredient is embedded in the matrix formed from the matrix forming natural compound.

In another embodiment, the composition contains pharmaceutically, nutraceutical and food approved excipients such as wetting agents, dispersing agents, glidants, flow property enhancers, preservatives, stabilizer, pH stabilizers, anti-oxidants etc.

In accordance with above embodiment, the slow or sustained or controlled release matrix releases the active ingredients by swelling or eroding or slowly dissolving or passive dissolution or microbial action in aqueous solutions or biological fluids. Preferably, the active ingredient, which requires slow release, is embedded or coated in the matrix during process of making the slow or sustained or controlled release composition. When said powdered or coated composition is added into water, the active ingredient is released slowly into water due to the swelling of matrix.

In accordance with the above embodiment, the active ingredient which requires slow release is coated with sustained release matrix during the process where coating can be single or multiple. Coating can be done using any coating equipments.

In another embodiment, the sustained release matrix can be used for coating the active ingredients, where coating can be single or double coating.

In further embodiment, the composition of the invention is used for wellbeing, preventing and treating human, animal and plant disease and also management of healthy lifestyle.

In an embodiment, the composition of the invention can be formulated into solid, liquid, suspension and semi-solid pharmaceutical, nutraceutical and veterinary formulations, oral formulation, topical formulations, ophthalmic formulations, otic formulations, oral suspensions, IV, IM, suppositories, etc.

In an embodiment, the composition of the invention can be combined with other technologies for therapeutic and safety benefits such as to improve bioavailability, improve the therapeutic effect, reducing the dose, dose frequency, to increase the patient compliance.

In another embodiment, the invention provides a process for manufacturing the sustained release formulation of the invention. The process in accordance with this embodiment comprises of following steps:
1. Adding required amount of a matrix forming natural compound in the form of oleoresins/oleo gum resin or gum resin or resin, spent, distillates, fatty acids, wax, lipid components or one of those mentioned in above matrix description in a reactor vessel;
2. Optionally, heating the mixture;
3. Optionally, adding organic acid;
4. Adding active ingredients either powder or granules;
5. Mixing the solution of step (4) for uniform distribution of the active ingredient;
6. Adding hardening agent such as metal hydroxides, metal oxides, metal carbonates and metal phosphates to the solution of step (5);
7. Mixing and hardening the solution or content of step (6) to get the solid mass;
8. Pulverise and Sieve the solid mass of step (7);
9. Optionally, adding excipients, and
10. Making into different dosage forms or filling in to HDPE containers or suitable packaging materials.

In an embodiment whereby the active ingredients are granular (above 1.5 mm) in nature, the process of invention comprises of:
1. Cleaning the coating equipment and adding required amount of the active ingredient;
2. Adding/spraying the pre-heated premix of liquid or semisolid matrix forming components such as oleoresins/oleo gum resin or gum resin or resin/lipids/fats/fatty acids/fatty acid distillates/wax/spent and optionally organic acid into the coating equipment of step (1);
3. Mixing well for uniform distribution of premix from step (2) on active ingredients;
4. Adding slowly/spraying metal hydroxides/oxides/carbonates/phosphates while mixing;
5. Mixing well to get the free flowing granules coated with matrix complex embedding active ingredients;
6. Repeating step (1) to (5) in case of double coating
7. Filling into different dosage forms, filling in to HDPE containers or into suitable packaging material.

In accordance with any of the above embodiments, the composition of invention can be prepared by using reactor, sigma mixers, mixers, conventional coating equipment, granulators, pelletizer, extractor, powder spraying equipment, etc.

### EXAMPLES

### Example 1: Composition of Lutein Sustained Release Formulation

TABLE I

| Sr. No. | Ingredients | Composition (g) |
|---|---|---|
| 1 | Turmeric Oleoresin | 63.73 |
| 2 | Propionic acid | 1.37 |
| 3 | Magnesium hydroxide | 4.90 |
| 4 | Precipitated silica | 2.00 |
| 5 | LUTEIN | 28.00 |

| | |
|---|---|
| Total | 100.00 |

### Process of Manufacturing Lutein Sustained Release Formulation:

1. Add turmeric oleoresin/spent (Liquid extract) in to the reactor vessel;
2. Optionally, heat the content in the reactor from the step (1);
3. Add required amount of propionic acid to step (2);
4. Mix for 10 mins to get uniform solution;
5. Add Lutein (active ingredient)
6. Mix the content of step (5) for uniform distribution of the Lutein;
7. Add Magnesium hydroxides to the solution of step (6);
8. Mix, transfer to SS tray and allow it to dry to get the solid mass;
9. Pulverize and Sieve the solid mass of step (8);
10. Add precipitated silica and other excipients
11. Fill into HDPE containers or capsules

### Example 2: Composition of Lutein Sustained Release Formulation

TABLE II

| Sr. No. | Ingredients | Composition (g) |
|---|---|---|
| 1 | Turmeric oleoresin | 65.06 |
| 2 | Propionic acid | 3.00 |
| 3 | HCl | 0.56 |
| 4 | Lutein | 25.00 |
| 5 | Lecithin (Deoiled) | 1.50 |
| 6 | Magnesium Hydroxide | 4.88 |

| | |
|---|---|
| Total | 100.00 |

### Sustained Release of Lutein:

Dissolution of Lutein was done by adding Lutein formulation at 1 mg/ml concentration in water and stirring continuously at 50 rpm. Sample was collected at regular intervals (0, 1, 2, 3, 4, 5 and 8 hrs) and analysed for Lutein content. Dissolution study results of Lutein formulation were compiled below.

### Leutin—HPLC Analysis:

Mobile phase: Hexane and ethyl acetate (3:1)
Standard solution: 150 μg/mL of USP Lutein RS in Mobile phase
Sample solution: Transfer 1 mL of the Sample stock 1.0% from the test for and evaporate under a stream of nitrogen to dryness. Add 1 mL of Mobile phase, and sonicate to dissolve.

### Detector: UV-Vis at 446 nm

Column: 4.6-mm×25-cm;
Flow rate: 1.5 mL/min
Injection size: 10 μL

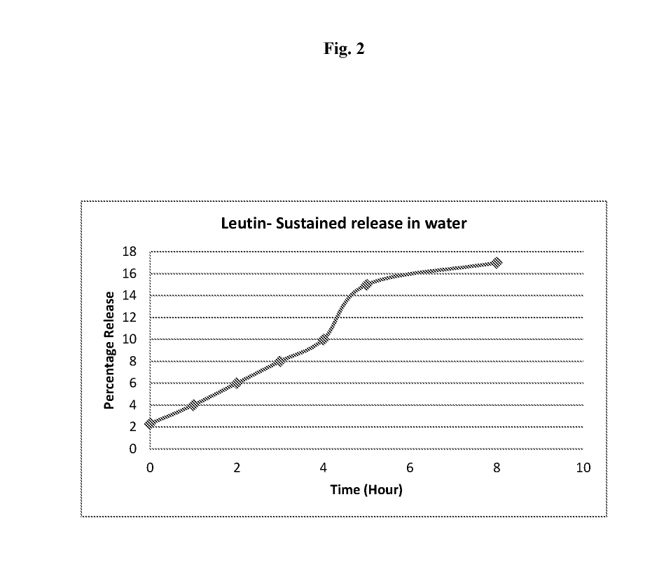

The Graph of the results obtained is shown in FIG. 2

### Example 3: Composition of Bacopa Sustained Release Formulation

TABLE III

| Sr. No. | Ingredients | Composition (g) |
|---|---|---|
| 1 | Turmeric Oleoresin | 65.70 |
| 2 | Propionic acid | 3.02 |
| 3 | Magnesium hydroxide | 4.78 |
| 4 | Precipitated silica | 2.00 |
| 5 | Bacopa monnieri extract | 24.50 |

| | |
|---|---|
| Total | 100.00 |

Process of Manufacturing Bacopa monnieri Sustained Release Formulation:
1. Add turmeric oleoresin/spent (Liquid extract) in to the reactor vessel;
2. Optionally, heat the content in the reactor from the step (1);
3. Add required amount of propionic acid to step (2);
4. Mix for 10 mins to get uniform solution;
5. Add Bacopa extract (active ingredient)
6. Mix the content of step (5) for uniform distribution of the Bacopa extract;
7. Add Magnesium hydroxides to the solution of step (6);
8. Mix, transfer to SS tray and allow it to dry to get the solid mass;
9. Pulverize and Sieve the solid mass of step (8);
10. Add precipitated silica and other excipients
11. Fill into HDPE containers or capsules

### Example 4: Composition of Caffeine Sustained Release Formulation

TABLE IV

| Sr. No. | Ingredients | Composition (g) |
|---|---|---|
| 1 | Turmeric oleoresin | 69.17 |
| 2 | Propionic acid | 3.33 |
| 3 | HCl | 0.40 |
| 4 | Caffeine | 20.00 |
| 5 | Lecithin (Deoiled) | 1.60 |
| 6 | Magnesium Hydroxide | 5.50 |

| | |
|---|---|
| Total | 100.00 |

### Sustained Release of Caffeine:

Dissolution of caffeine was done by adding Caffeine formulation at 1 mg/ml concentration in water and stirring continuously at 50 rpm. Sample was collected at regular intervals (0, 1, 2, 3, 4, 5 and 8 hrs) and analysed for caffeine content. Dissolution study results of caffeine formulation were compiled below.

### Caffeine—HPLC Analysis:

Mobile phase: Acetonitrile, tetrahydrofuran, and Buffer (25:20:955). Adjust with glacial acetic acid to a pH of 4.5.
Buffer: 0.82 g/L of anhydrous sodium acetate
Standard solution: 0.5 mg of USP Caffeine dissolved in 25 ml of Mobile phase

### Detector: UV—275 nm

Column: 4.6-mm×15-cm;
Flow rate: 1 mL/min;
Injection volume: 10 μL.

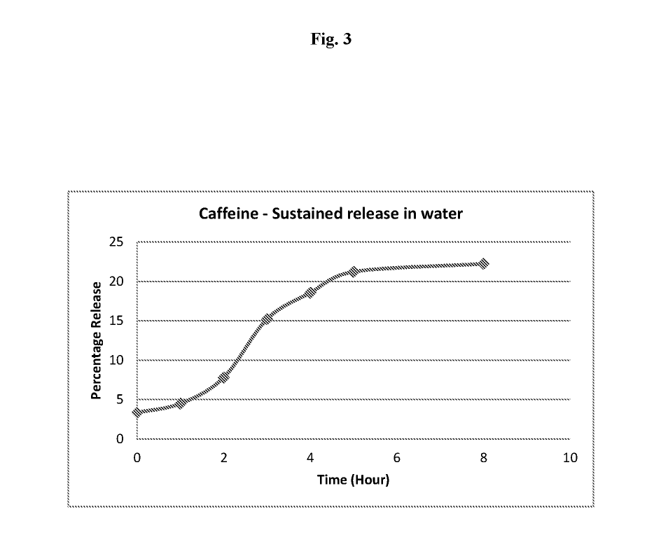

The graph of the result so obtained is plotted as graph presented in FIG. 3

### Example 5: Composition of Vitamin B6 (Pyridoxine HCl) Sustained Release Formulation

TABLE V

| Sr. No. | Ingredients | Composition (g) |
|---|---|---|
| 1 | Turmeric oleoresin | 69.18 |
| 2 | Propionic acid | 3.32 |
| 3 | HCl | 0.40 |
| 4 | Pyridoxine | 20.00 |
| 5 | Lecithin (Deoiled) | 1.60 |
| 6 | Magnesium Hydroxide | 5.50 |

| | |
|---|---|
| Total | 100.00 |

### Sustained Release of Vitamin B6:

Dissolution of caffeine was done by adding Vitamin B6 formulation at 1 mg/ml concentration in water and stirring continuously at 50 rpm. Sample was collected at regular intervals (0, 1, 2, 3, 4, 5 and 8 hrs) and analysed for Vitamin B6 content. Dissolution study results of Vitamin B6 formulation were compiled below.

### Vitamin B6—HPLC Analysis:

Mobile phase: Mix 10 mL of glacial acetic acid, 0.6 g of sodium-1-hexanesulfonate, and 700 mL of water in a 1000-mL volumetric flask. Adjust with glacial acetic acid or 1 N sodium hydroxide to a pH of 3.0. Add 235 mL of methanol, and dilute with water to volume.
Standard solution: 5 mg/mL of p-hydroxybenzoic acid solution in mobile phase. Prepare a 0.5-mg/mL solution of USP Pyridoxine Hydrochloride RS in Mobile phase. Transfer 10.0 mL of this solution and 1.0 mL of internal standard to a 100-mL volumetric flask, and dilute with mobile phase.

### Detector: UV—280 nm

Column: 4.6-mm×15-cm;
Flow rate: 1.5 mL/min
Injection volume: 10 μL

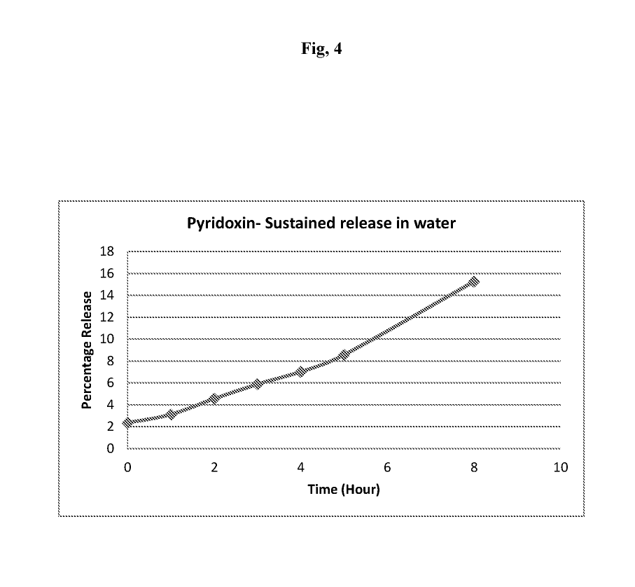

The graph of the result so obtained is plotted as graph presented in FIG. 4.

### Example 6: Composition of Vitamin C (Ascorbic Acid) Sustained Release Formulation

TABLE VI

| Sr. No. | Ingredients | Composition (g) |
|---|---|---|
| 1 | Turmeric oleoresin | 69.18 |
| 2 | Propionic acid | 3.32 |
| 3 | HCl | 0.40 |
| 4 | Ascorbic acid | 20.00 |
| 5 | Lecithin (Deoiled) | 1.60 |
| 6 | Magnesium Hydroxide | 5.50 |

| | |
|---|---|
| Total | 100.00 |

### Sustained Release of Vitamin C:

Dissolution of caffeine was done by adding Vitamin C formulation at 1 mg/ml concentration in water and stirring continuously at 50 rpm. Sample was collected at regular intervals (0, 1, 2, 3, 4, 5 and 8 hrs) and analysed for Vitamin C content. Dissolution study results of Vitamin C formulation were compiled below.

### Vitamin C—HPLC Analysis:

Mobile phase: 2.04 g/L of monobasic potassium phosphate in water. Adjust with phosphoric acid to a pH of 3.0.
Standard solution: 0.25 mg/mL of USP Ascorbic Acid RS in Diluent.
Diluent: 0.56 g of edetate disodium dihydrate and 2.04 g of monobasic potassium phosphate per 1000 mL of water. Adjust with phosphoric acid to a pH of 3.0.

### Detector: UV—245 nm

Column: 4.6-mm×15-cm;
Flow rate: 1 mL/min
Injection volume: 5 μL

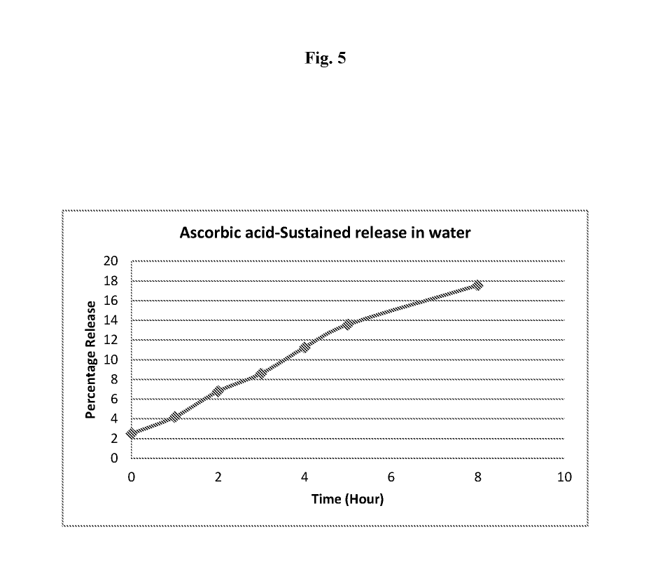

The graph of the result so obtained is plotted as graph presented in FIG. 5.

### Example 7: Composition of Zeaxanthin Sustained Release Formulation

TABLE VII

| Sr. No. | Ingredients | Composition (g) |
|---|---|---|
| 1 | Turmeric oleoresin | 69.18 |
| 2 | Propionic acid | 3.32 |
| 3 | HCl | 0.40 |
| 4 | Zeaxanthin | 20.00 |
| 5 | Lecithin (Deoiled) | 1.60 |
| 6 | Magnesium Hydroxide | 5.50 |

| | |
|---|---|
| Total | 100.00 |

### Sustained Release of Zeaxanthin:

Dissolution of caffeine was done by adding Zeaxanthin formulation at 1 mg/ml concentration in water and stirring continuously at 50 rpm. Sample was collected at regular intervals (0, 1, 2, 3, 4, 5 and 8 hrs) and analysed for Zeaxanthin content. Dissolution study results of Zeaxanthin formulation were compiled below.

### Zeaxanthin—HPLC Analysis:

Mobile phase: Hexane and ethyl acetate (3:1)
Standard solution: 150 μg/mL of USP Zeaxanthine in Mobile phase
Sample solution: Transfer 1 mL of the Sample stock 1.0% from the test for

Content of Total Carotenoids, and evaporate under a stream of nitrogen to dryness.

Add 1 mL of Mobile phase, and sonicate to dissolve.

### Detector: UV-V is at 446 nm

Column: 4.6-mm×25-cm;
Flow rate: 1.5 mL/min

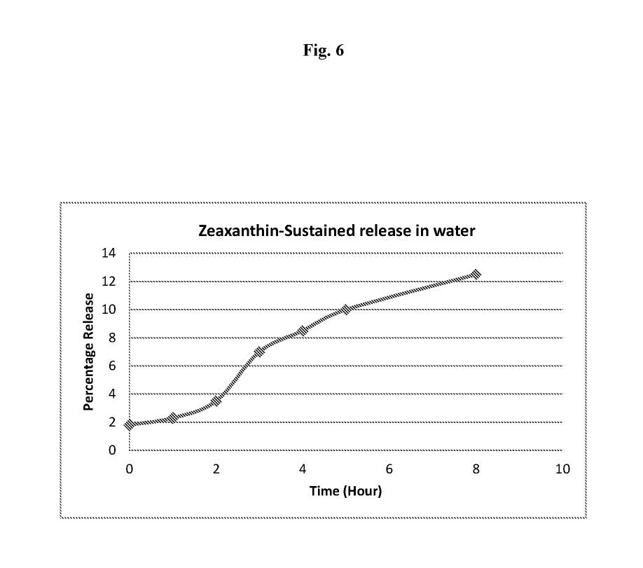

The graph of the result so obtained is plotted as graph presented in FIG. 6

### Example 8: Composition of Silymarin (Milk Thistle Extract) Sustained Release Formulation

TABLE VIII

| Sr. No. | Ingredient | Composition (g) |
|---|---|---|
| 1 | Turmeric oleoresin | 69.18 |
| 2 | Propionic acid | 3.32 |
| 3 | HCl | 0.40 |
| 4 | Silymarin | 20.00 |
| 5 | Lecithin (Deoiled) | 1.60 |
| 6 | Magnesium Hydroxide | 5.5 |

| | |
|---|---|
| Total | 100.00 |

Dissolution of caffeine was done by adding Silymarin formulation at 1 mg/ml concentration in water and stirring continuously at 50 rpm. Sample was collected at regular intervals (0, 1, 2, 3, 4, 5 and 8 hrs) and analysed for Silymarin content.

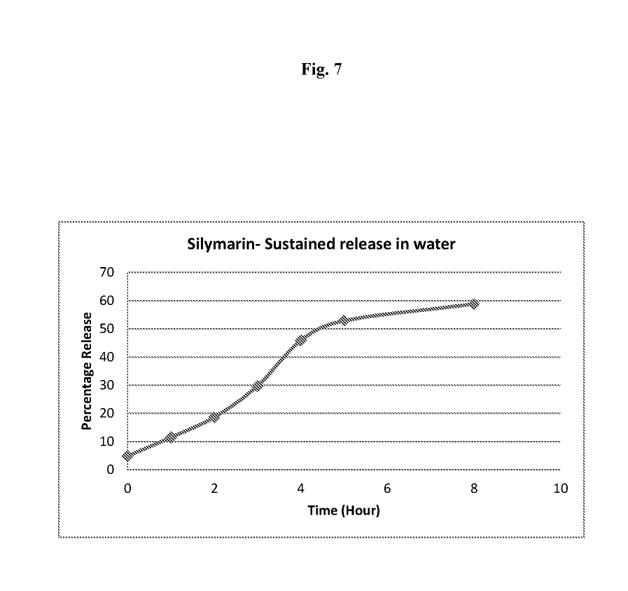

The graph of the result so obtained is plotted as graph presented in FIG. 7

### Example 9: Composition of Chlorogenic Acid Sustained Release Formulation

TABLE IX

| Sr. No. | Ingredient | Composition (g) |
|---|---|---|
| 1 | Turmeric oleoresin | 69.176 |
| 2 | Propionic acid | 3.328 |
| 3 | HCl | 0.4 |
| 4 | Chlorogenic acid | 20.0 |
| 5 | Lecithin | 1.6 |
| 6 | Magnesium Hydroxide | 5.496 |

| | |
|---|---|
| Total | 100.0 |

Dissolution of caffeine was done by adding Chlorogenic acid formulation at 1 mg/ml concentration in water and stirring continuously at 50 rpm. Sample was collected at regular intervals (0, 1, 2, 3, 4, 5 and 8 hrs) and analysed for Chlorogenic acid content.

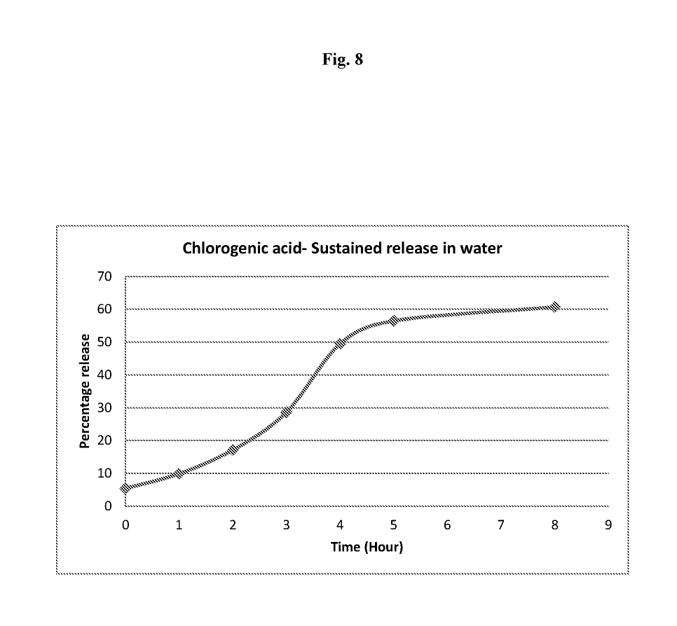

The graph of the result so obtained is plotted as graph presented in FIG. 8

### Example 10: Composition of Bacosides Sustained Release Formulation

TABLE X

| Sr. No. | Ingredient | Composition (g) |
|---|---|---|
| 1 | Turmeric oleoresin | 69.176 |
| 2 | Propionic acid | 3.328 |
| 3 | HCl | 0.4 |
| 4 | Bacoside | 20 |
| 5 | Lecithin (Deoiled) | 1.6 |
| 6 | Magnesium Hydroxide | 5.496 |

| | |
|---|---|
| Total | 100 |

Dissolution of caffeine was done by adding Bacosides formulation at 1 mg/ml concentration in water and stirring continuously at 50 rpm. Sample was collected at regular intervals (0, 1, 2, 3, 4, 5 and 8 hrs) and analysed for Bacosides content.

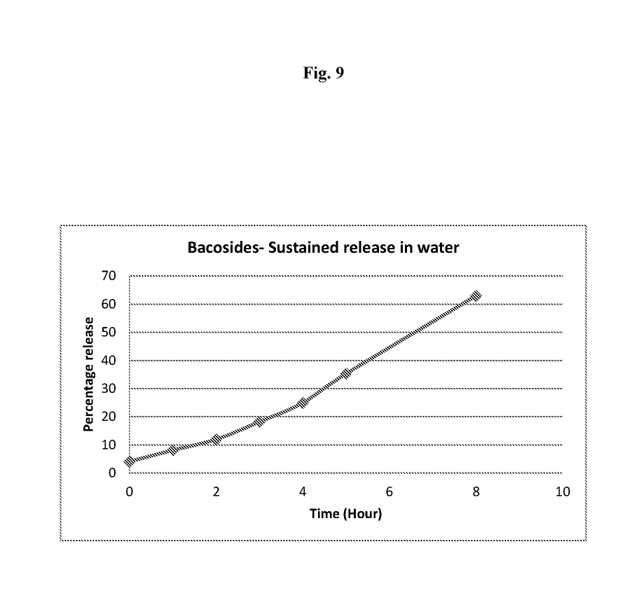

The graph of the result so obtained is plotted as graph presented in FIG. 9

### Example 11: Composition of Policosanals Sustained Release Formulation

TABLE XI

| Sr. No. | Ingredient | Composition (g) |
|---|---|---|
| 1 | Turmeric oleoresin | 69.176 |
| 2 | Propionic acid | 3.328 |
| 3 | HCl | 0.4 |
| 4 | Policosonals | 20 |
| 5 | Lecithin (Deoiled) | 1.6 |
| 6 | Magnesium Hydroxide | 5.496 |

| | |
|---|---|
| Total | 100 |

Dissolution of caffeine was done by adding Policosonals formulation at 1 mg/ml concentration in water and stirring continuously at 50 rpm. Sample was collected at regular intervals (0, 1, 2, 3, 4, 5 and 8 hrs) and analysed for Policosonals content.

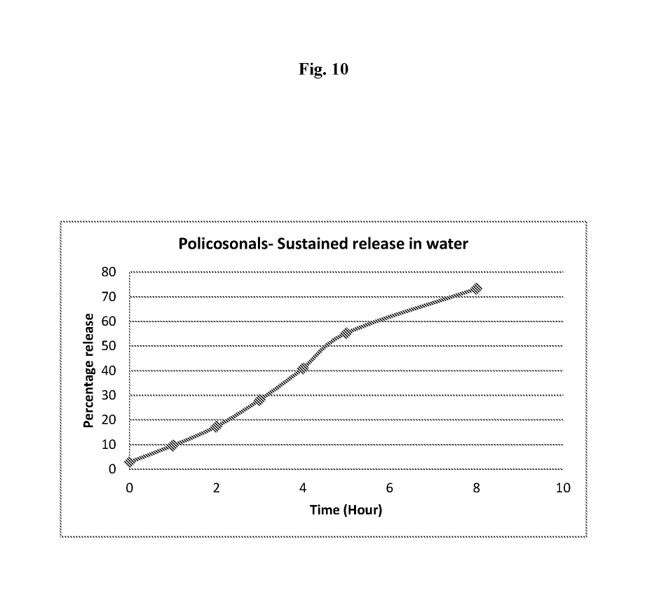

The graph of the result so obtained is plotted as graph presented in FIG. 10

### Example 12: Composition of Astaxanthin Sustained Release Formulation

TABLE XII

| Sr. No. | Ingredient | Composition (g) |
|---|---|---|
| 1 | Turmeric oleoresin | 69.18 |
| 2 | Propionic acid | 3.32 |
| 3 | HCl | 0.40 |
| 4 | Astaxanthin | 20.00 |
| 5 | Lecithin (Deoiled) | 1.60 |
| 6 | Magnesium Hydroxide | 5.5 |
| | Total | 100.00 |

Dissolution of caffeine was done by adding Astaxanthin formulation at 1 mg/ml concentration in water and stirring continuously at 50 rpm. Sample was collected at regular intervals (0, 1, 2, 3, 4, 5 and 8 hrs) and analysed for Astaxanthin content.

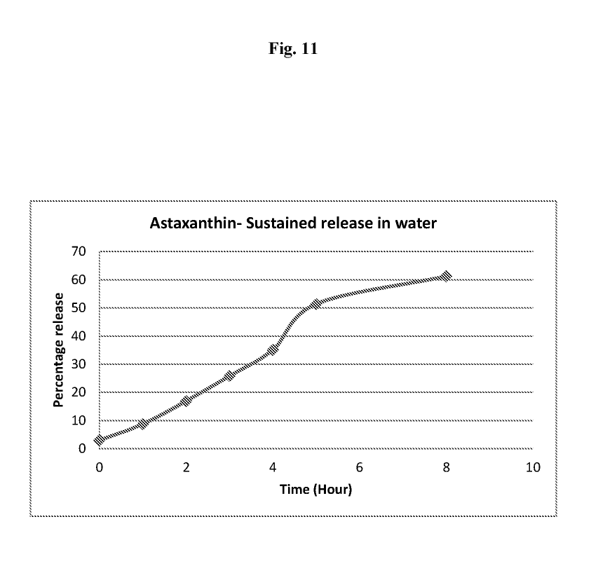

The graph of the result so obtained is plotted as graph presented in FIG. 11

### Example 13: Composition of Urea Sustained Release Matrix Formulation

TABLE XIII

| Sr. No. | Ingredients | Composition (g) |
|---|---|---|
| 1 | Turmeric Oleoresin spent | 42.14 |
| 2 | Propionic acid | 1.47 |
| 3 | Magnesium hydroxide | 5.39 |
| 4 | Urea (Powder) | 49.00 |
| 5 | Silicon dioxide | 2.00 |

| | |
|---|---|
| Total | 100.00 |

### Process of Manufacturing Urea Sustained Release Formulation:

1. Add turmeric oleoresin/spent (Liquid extract) in to the reactor vessel;
2. Optionally, heat the content in the reactor from the step (1);
3. Add required amount of propionic acid to step (2);
4. Mix for 10 mins to get uniform solution;
5. Add Urea (granules or powder)
6. Mix the content of step (5) for uniform distribution of the Urea;
7. Add Magnesium hydroxides to the solution of step (6);
8. Mix, transfer to SS tray and allow it to dry to get the solid mass;
9. Pulverize and Sieve the solid mass of step (8);
10. Add precipitated silica and other excipients
11. Fill into HDPE containers or capsules

### Example 14: Composition of Boswellia Sustained Release Formulation

TABLE XIV

| | Ingredients | Composition (g) |
|---|---|---|
| 1 | Turmeric Oleoresin | 66.82 |
| 2 | Propionic acid | 2.48 |
| 3 | Magnesium hydroxide | 9.9 |
| 4 | Precipitated silica | 1 |
| 5 | Boswellia serrata extract | 19.8 |

| | |
|---|---|
| Total | 100 |

Process of Manufacturing Boswellia serrata Extract Sustained Release Formulation:
1. Add turmeric oleoresin (Liquid extract) in to the reactor vessel;
2. Optionally, heat the content in the reactor from the step (1);
3. Add required amount of propionic acid to step (2);
4. Mix for 10 mins to get uniform solution;
5. Add Boswellia serrata extract (active ingredient);
6. Mix the content of step (5) for uniform distribution of the Boswellia serrata extract;
7. Add Magnesium hydroxides to the solution of step (6);
8. Mix, transfer to SS tray and allow it to dry to get the solid mass;
9. Pulverize and Sieve the solid mass of step (8);
10. Add precipitated silica and other excipients, and
11. Fill into HDPE containers or capsules.

### Example 15: Composition of Urea Sustained Release Formulation

TABLE XV

| | Ingredients | Composition (g) |
|---|---|---|
| 1 | Turmeric Oleoresin | 66.82 |
| 2 | Propionic acid | 2.48 |
| 3 | Magnesium hydroxide | 9.9 |
| 4 | Precipitated silica | 1 |
| 5 | Urea (granules) | 19.8 |

| | |
|---|---|
| Total | 100 |

Process of Manufacturing Slow Release Urea Formulation with Coating Equipment
1. Add Urea granules to the coating equipment and start the machine;
2. Slowly add or spray the premix of turmeric oleoresin spent and propionic acid into the coating equipment of step (1);
3. Mixing well for uniform distribution from step (2);
4. Add slowly/spray magnesium hydroxide while mixing;
5. Mixing well to get the free flowing coated granules;
6. add optional excipients and
7. Fill into different HDPE containers or into suitable packaging material.

### Example 16

### Composition of Urea Sustained Release Formulation.

TABLE XVI

| Sr. No. | Ingredients | Composition (g) |
|---|---|---|
| 1 | Urea | 70.0 |
| 2 | Palm fatty acid distillate | 15.0 |
| | (PFDA)/Rice bran wax/Palm | |
| | stearin/fatty acid residue | |
| 3 | Calcium hydroxide | 15.0 |

| | |
|---|---|
| Total | 100.0 |

Process of Manufacturing Slow Release Urea Formulation with Coating Equipment
1. Add Urea granules to the coating equipment and start the machine
2. Slowly add Add/spray the preheated palm fatty acid distillate to step (1);
3. Mix well for uniform distribution from step (2);
4. Add slowly/spray calcium hydroxide while mixing;
5. Mixing well to get the free flowing coated granules;
6. sieve to remove excess calcium hydroxide
7. Fill into different HDPE containers or into suitable packaging material.

### Example 17

### Composition of Urea Sustained Release Formulation: Double Coated Urea Formulation

TABLE XVII
Step a: First Coating

| | | Composition (g) | Composition (g) |
|---|---|---|---|
| | Ingredients | OLVP-01 (a) | OLVP-01 (a) |
| 1 | Urea | 80.0 | 87.5 |
| 2 | Rice bran wax/PFAD/Palm | 10.0 | 5.0 |
| | stearine/Fatty acid residue | | |
| 3 | Calcium hydroxide | 10.0 | 7.5 |

| | | |
|---|---|---|
| Total | 100.0 | 100.0 |

### Process of Manufacturing Slow Release Urea Formulation

1. Add Urea granules to the coating equipment and start the machine
2. Slowly add Add/spray the preheated Rice bran wax/PFAD/Palm stearine/Fatty acid residue to step (1);
3. Mix well for uniform distribution from step (2);
4. Add slowly/spray calcium hydroxide while mixing;
5. Mix well to get the free flowing coated granules;
6. Sieve to remove excess calcium hydroxide
7. Fill into different HDPE containers or into suitable packaging material.

### Step b.: Second Coating—

TABLE XVIII

| | | Composition (g) | Composition (g) |
|---|---|---|---|
| | | Formulation | Formulation |
| | Ingredients | OLVP-01 (b) | OLVP-02 (b) |
| 1 | Urea from Step (b) | 80.0 | 87.5 |
| | (Single coated) | | |
| 2 | Rice Bran Wax/PFAD/Palm | 10.0 | 6.25 |
| | stearine/Fatty acid residue | | |
| 3 | Calcium hydroxide | 10.0 | 6.25 |

| | | |
|---|---|---|
| Total | 100.0 | 100.0 |

1. Add single coated Urea granules to the coating equipment and start the machine
2. Slowly add Add/spray the preheated Rice Bran wax/PFAD/Palm stearine/Fatty acid residue to step (1);
3. Mix well for uniform distribution from step (2);
4. Add slowly/spray calcium hydroxide while mixing;
5. Mix well to get the free flowing coated granules;
6. Sieve to remove excess calcium hydroxide
7. Fill into different HDPE containers or into suitable packaging material.

### Final Composition:

TABLE XIX

| | | Composition (g) | Composition (g) |
|---|---|---|---|
| | | Formulation | Formulation |
| | Ingredients | OLVP-01 | OLVP-02 |
| 1 | Urea | 64.0 | 76.56 |
| 2 | Rice Bran Wax/PFAD/Palm | 18.0 | 10.63 |
| | stearine/Fatty acid residue | | |
| 3 | Calcium hydroxide | 18.0 | 12.81 |

| | | |
|---|---|---|
| Total | 100.0 | 100.0 |

### Example 18: Urea Release Results for Product from Example 17 (Formulation OLVP-01)

Urea release from the double coated urea in comparison to Optigen: Known amount of urea product was added into known amount of water and urea release into the water was analysed over a time of period using standardised UV-Visible spectrophotometer method.

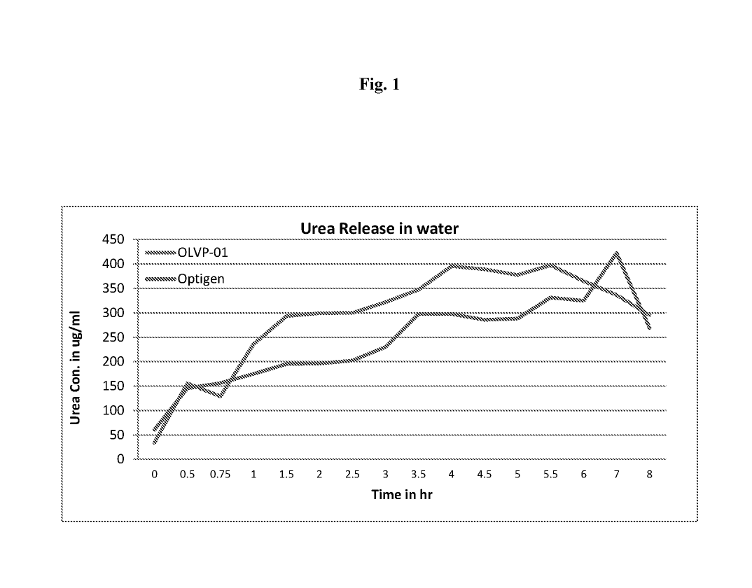

The results obtained are plotted in form of graph of FIG. 1.

### Example 19: Composition of Berberin Sustained Release Formulation

TABLE XX

| Sr. No. | Ingredient | Composition(g) |
|---|---|---|
| 1 | Turmeric oleoresin | 69.18 |
| 2 | Propionic acid | 3.33 |
| 3 | HCl | 0.40 |
| 4 | Berberin | 20.00 |
| 5 | Lecithin | 1.60 |
| 6 | Magnesium Hydroxide | 5.50 |

| | |
|---|---|
| Total | 100.00 |

### Example 20: Composition of Methyl Cobalamine Sustained Release Formulation

TABLE XXI

| Sr. No. | Ingredient | Composition (g) |
|---|---|---|
| 1 | Turmeric oleoresin | 69.18 |
| 2 | Propionic acid | 3.33 |
| 3 | HCl | 0.40 |
| 4 | Methyl cobalamine | 20.00 |
| 5 | Lecithin | 1.60 |
| 6 | Magnesium Hydroxide_5.50 | |

| | |
|---|---|
| Total | 100.00 |

### Example 21: Composition of Folic Acid Sustained Release Formulation

TABLE XXII

| Sr. No. | Ingredient | Composition (g) |
|---|---|---|
| 1 | Turmeric oleoresin | 69.18 |
| 2 | Propionic acid | 3.33 |
| 3 | HCl | 0.40 |
| 4 | Follic acid | 20.00 |
| 5 | Lecithin | 1.60 |
| 6 | Magnesium Hydroxide | 5.50 |

| | |
|---|---|
| Total | 100.00 |

### Example 22: Composition of Magnesium Sustained Release Formulation

TABLE XXIII

| Sr. No. | Ingredient | Composition (g) |
|---|---|---|
| 1 | Turmeric oleoresin | 69.18 |
| 2 | Propionic acid | 3.33 |
| 3 | HCl | 0.40 |
| 4 | Magnesium Sulphate | 20.00 |
| 5 | Lecithin | 1.60 |
| 6 | Magnesium Hydroxide | 5.50 |

| | |
|---|---|
| Total | 100.00 |

### Example 23: Composition of Calcium Sustained Release Formulation

TABLE XXIV

| Sr. No. | Ingredient | Composition (g) |
|---|---|---|
| 1 | Turmeric oleoresin | 69.18 |
| 2 | Propionic acid | 3.33 |
| 3 | HCl | 0.40 |
| 4 | Di calcium phosphate 95% | 20.00 |
| 5 | Lecithin | 1.60 |
| 6 | Magnesium Hydroxide | 5.50 |

| | |
|---|---|
| Total | 100.00 |

### Example 24: Composition of Iron Sustained Release Formulation

TABLE XXV

| Sr. No. | Ingredient | Composition (g) |
|---|---|---|
| 1 | Turmeric oleoresin | 69.18 |
| 2 | Propionic acid | 3.33 |
| 3 | HCl | 0.40 |
| 4 | Ferrous sulphate | 20.00 |
| 5 | Lecithin | 1.60 |
| 6 | Magnesium Hydroxide | 5.50 |

| | |
|---|---|
| Total | 100.00 |

### Example 25: Composition of Pterostilbene Sustained Release Formulation

TABLE XXVI

| Sr. No. | Ingredient | Composition (g) |
|---|---|---|
| 1 | Turmeric oleoresin | 69.18 |
| 2 | Propionic acid | 3.33 |
| 3 | HCl | 0.40 |
| 4 | pterostilbene | 20.00 |
| 5 | Lecithin | 1.60 |
| 6 | Magnesium Hydroxide | 5.50 |

| | |
|---|---|
| Total | 100.00 |

1. A composition for slow/sustained/controlled release of at last one active ingredient comprising:
a. at least one active ingredient in the range of 0.1 to 99%;
b. at least one matrix forming or coat forming natural compound in the range of 2 to 98%, and
c. at least one hardening agent in the range of 1 to 90%.

2. The composition of claim 1, wherein the active ingredient is selected from the group consisting of pharmaceuticals, phytochemicals, nutraceuticals, phytochemical containing components, vitamins, metals, plant extract, animal extract, food ingredients, beverage ingredients, veterinary drugs, veterinary supplement, veterinary additives, agrifood ingredients, and agricultural ingredients.

3. The composition in accordance with claim 2, wherein the matrix forming or coating forming natural compound is selected from the group consisting of oleoresins, oleoresin spent, oleo gum resins, gum resins, resins, rosins, lipids, fats, fatty acids, fatty acid distillates, fatty acid residues, palm stearine, sludge oil, fatty acid derivatives, spent liquor, mother liquor, wax or by-products of food and agricultural industries.

4. The composition in accordance with claim 1, wherein the hardening agent is selected from the group consisting of metal hydroxides, metal oxides, metal chlorides and metal carbonates, and combinations thereof.

5. The composition in accordance with claim 4, wherein the hardening agent is selected from the group consisting of Calcium Hydroxide (Ca(OH)₂), Calcium Oxide (CaO), Magnesium hydroxide (Mg(OH)₂), Magnesium Oxide (MgO), Magnesium Chloride (MgCl₂), Zinc Hydroxide (Zn(OH)₂), Zinc Oxide (ZnO), Iron hydroxide (Fe(OH)₂), Iron Oxide (FeO), Calcium Chloride (CaCl₂), Calcium carbonate (CaCO₃), Magnesium chloride (MgCl₂), Magnesium carbonate (MgCO₃), dicalcium phosphate (CaHPO₄), sodium hydroxide (NaOH), potassium hydroxide (KOH), and combinations thereof.

6. The composition in accordance with claim 5, wherein the hardening agent is selected from the group consisting of Calcium Hydroxide (Ca(OH)₂), Magnesium Hydroxide (Mg(OH)₂), and combination thereof.

7. The composition in accordance with claim 1, wherein the composition further comprises an acid.

8. The composition in accordance with claim 7, wherein the acid is selected from the group consisting of propionic acid, citric acid, fumaric acid, succinic acid, maleic acid, tartaric acid, adipic acid, acetic acid, hydrochloric acid, sulphuric acid, phosphoric acid and combinations thereof.

9. The process for manufacturing the composition for slow/sustained/controlled release of active in accordance with claim 1, said process comprising:
a. adding a desired amount of the matrix forming or coat forming natural compound to a reactor vessel;
b. adding powder or granules of the active ingredient in the reactor vessel;
c. mixing the contents of the reactor vessel of step (b) for uniform distribution of the active ingredient to obtain a solution;
d. adding the hardening agent to the solution of step (c), mixing for 5 to 60 minutes and transferring the contents of the reaction vessel to stainless steel trays;
e. allowing the transferred contents of step (d) to harden into a solid mass in the stainless steel trays;
f. pulverising and sieving the solid mass obtained in step (e);
g. adding desired excipients to the pulverised mass of step (f), and
h. preparing a dosage form from the pulverised mass of step (h).

10. The process of claim 9, wherein the process further comprises:
heating the matrix forming or coat forming natural compound of step (a) of claim 9 to a suitable temperature, and
adding a desired amount of an acid to the reaction vessel of step (a) and continuing to heat at the suitable temperature.

11. The process for manufacturing the slow release matrix composition of claim 1, comprising:
a. adding a desired amount of a granular active ingredient to coating equipment;
b. adding or spraying a premix of preheated liquid or semisolid matrix forming or coat forming natural compound and an optional acid into the coating equipment of step (a);
c. mixing the content of step (b) for uniform distribution of the premix on the granular active ingredient;
d. adding or spraying the hardening agent into the coating equipment while mixing the content of step (c);
e. Mixing the content of step (d) to obtain free flowing granules uniformly coated with a matrix complex embedding the active ingredient;
f. repeating steps (a) to (e) to obtain double coated free flowing granules with matrix complex embedding active ingredient, and
g. packing the coated free flowing granules of step (e) and/or step (f) in desired dosage form.

12. The process of claim 11, wherein natural or synthetic colouring agents are used to give the desired colour to the final product.

13. The process of claim 9, wherein the matrix forming or coating forming natural compound is selected from the group consisting of oleoresins, oleoresin spent, oleo gum resins, gum resins, resins, rosins, lipids, fats, fatty acids, fatty acid distillates, fatty acid residues, palm stearine, sludge oil, fatty acid derivatives, spent liquor, mother liquor, wax or by-products of food and agricultural industries.

14. The process of claim 9, wherein the acid is selected from the group consisting of propionic acid, citric acid, fumaric acid, succinic acid, maleic acid, tartaric acid, adipic acid, acetic acid, hydrochloric acid, sulphuric acid, phosphoric acid and combinations thereof.

15. The process of claim 9 wherein the hardening agent is selected from the group consisting of Calcium Hydroxide (Ca(OH)₂), Calcium Oxide (CaO), Magnesium hydroxide (Mg(OH)₂), Magnesium Oxide (MgO), Magnesium Chloride (MgCl₂), Zinc Hydroxide (Zn(OH)₂), Zinc Oxide (ZnO), Iron hydroxide (Fe(OH)₂), Iron Oxide (FeO), Calcium Chloride (CaCl₂), Calcium carbonate (CaCO₃), Magnesium chloride (MgCl₂), Magnesium carbonate (MgCO₃), dicalcium phosphate (CaHPO₄), sodium hydroxide (NaOH), potassium hydroxide (KOH), and combinations thereof.

16. A slow release formulation for urea comprising,
a. urea;
b. a natural coating component, and
c. a hardening agent.

17. The slow release formulation of claim 16, wherein the formulation further comprises an organic or inorganic acid.

18. The slow release formulation of claim 16, wherein the organic acid is selected from the group consisting of propionic acid, citric acid, fumaric acid, succinic acid, maleic acid tartaric acid, adipic acid, acetic acid

19. The slow release formulation of claim 17, wherein the natural coating component is solid, semisolid or liquid.

20. The slow release formulation of claim 18, wherein the natural coating component is selected from the group consisting of an oleoresin, oleoresin spent, an oleo gum resin, a gum resin, a resin, rosin, a lipid, a hydrogenated or non-hydrogenated fat, a hydrogenated or non-hydrogenated fatty acid, fatty acid distillates, fatty acid residue, Palm stearin, sludge oil, fatty acid derivatives, spent, mother liquors and/or a wax (plant and/or animal waxes) or by-products of food and agricultural industries.

21. The slow release formulation of claim 16, wherein the hardening agent is a metal hydroxide or a metal oxide or a metal chloride or a metal carbonate or a combination thereof.

22. The slow release formulation of claim 20, wherein the hardening agent is Calcium Hydroxide (Ca(OH)₂), Magnesium Hydroxide (Mg(OH)₂), or a combination thereof.

23. The slow release formulation of claim 16, wherein the process for manufacturing said formulation comprises of:
a. adding granular urea to a coating vessel and start the vessel at 5 to 100 RPM
b. heating the granular urea in the coating vessel to 50° C. while mixing at 5 to 100 RPM
c. adding a desired amount of preheated natural coating components (coating material) and continue mixing till the temperature comes down to approximately 46° C.;
d. Adding calcium hydroxide to the vessel content and mixing it well to get free flowing granules of coated urea; and
e. Packing the final coated urea into a suitable packing material.

24. The slow release formulation in accordance with claim 16, wherein the slow release formulation further comprises natural or synthetic colouring agents for giving colour to final granules.
